(19) United States
(12) Patent Application Publication
(10) Pub. No.: US 2016/0287626 A1
(43) Pub. Date: Oct. 6, 2016

(54) METHODS OF TREATING AND PREVENTING RADIATION DAMAGE

(71) Applicant: CANTEX PHARMACEUTICALS, INC., Weston, FL (US)

(72) Inventor: Stephen MARCUS, Weston, CA (US)

(21) Appl. No.: 15/031,248

(22) PCT Filed: Oct. 21, 2014
(86) PCT No.: PCT/US2014/061634

§ 371 Date: Apr. 21, 2016

Related U.S. Application Data
(60) Provisional application No. 61/894,343, filed on Oct. 22, 2013.

(51) Int. Cl. A61K 31/727

(52) U.S. Cl. CPC A61K 31/727

(57) ABSTRACT

The invention relates to methods of treating and preventing radiation damage from whole-body exposure. According to the methods of the invention, subjects are treated therapeutically and/or prophylactically with low-anticoagulant heparinoids. The invention also relates to methods of extending the life of subjects exposed to whole-body radiation.

### FIELD OF THE INVENTION

The invention relates to methods of treating and preventing radiation damage caused by whole-body radiation exposure.

### BACKGROUND OF THE INVENTION

In the event of a nuclear attack or damage to a nuclear reactor, a large number of people could be exposed to whole-body radiation, at various doses, and therefore put at risk of developing some degree of acute radiation syndrome (ARS). ARS, also known colloquially as radiation poisoning, is a constellation of health effects which present shortly after a subject is exposed to high levels of ionizing radiation. ARS is initially characterized by headache, nausea, and vomiting but can progress to hematological, gastrointestinal, neurological, pulmonary, and other major organ dysfunction.

The degree of symptom severity and prognosis of ARS is directly correlated to the absorbed dose of radiation. The LD_{50/60} (fatal dose to 50% of subjects within 60 days) for whole-body radiation is about 3 Gy. With medical care, e.g., antibiotics, blood transfusions, bone marrow transplants, some subjects may survive ARS with exposures up to 6 Gy and occasionally higher. Exposure greater than 10 Gy generally results in death within 1-2 weeks of exposure.

For those subjects who survive the acute effects of radiation exposure, whole-body radiation exposure also produces delayed radiation effects such as lifespan shortening, cataract development, and carcinogenesis, which can occur months to decades later. Chronic radiation syndrome also presents a constellation of health effects that occur after months or years of chronic exposure to high amounts of ionizing radiation.

At present, no approved drugs are available for the prevention or treatment of radiation damage associated with whole-body radiation. There is, therefore, an urgent need to develop therapies for subjects suffering the effects of whole-body radiation exposure, and those at risk of whole-body radiation exposure.

### SUMMARY OF THE INVENTION

In a first aspect, methods of treating or preventing radiation damage in a subject exposed to whole-body radiation are presented, comprising administering to a subject exposed to whole-body radiation a therapeutically or prophylactically effective amount of a low-anticoagulant heparinoid. In a second aspect, methods are presented for extending the life of a subject exposed to whole-body radiation, comprising administering to a subject exposed to whole-body radiation a therapeutically or prophylactically effective amount of a low-anticoagulant heparinoid.

In certain embodiments, the low-anticoagulant heparinoid of the invention has an average molecular weight of about 8 kDa to about 15 kDa. The low-anticoagulant heparinoid may be desulfated or substantially desulfated at the 2-O position or the 3-O position. In certain embodiments, the low-anticoagulant heparinoid is desulfated or substantially desulfated at both the 2-O position and the 3-O position. In particular embodiments, the low-anticoagulant heparinoid is ODSH, which is described in greater detail herein below.

The low-anticoagulant heparinoid may be administered parenterally. In particular embodiments, the low-anticoagulant heparinoid is administered intravenously and/or subcutaneously.

The low-anticoagulant heparinoid may be administered prior to, and/or during, and/or following exposure to whole-body radiation. In certain embodiments, the subject is administered the low-anticoagulant heparinoid following exposure to whole-body radiation, such as within 60 hours after exposure to whole-body radiation. The subject may be administered the low-anticoagulant heparinoid immediately following, or about 2 hours or more after exposure to whole-body radiation.

In certain embodiments, a subject is administered the low-anticoagulant heparinoid prior to exposure to whole-body radiation.

The methods described herein may be used for subjects exposed to whole-body radiation at a dose of about 0.1 Gy/min or greater, such as about 0.5 Gy/min or greater. In certain embodiments, the subject has received a whole-body absorbed dose of radiation about 2 Gy or greater, such as about 6 Gy or greater or even about 8 Gy or greater. The whole-body radiation of a subject may occur over a time period of about 2 hours or less, such as about 1 hour or less.

The low-anticoagulant heparinoid may be administered in one or more doses, such as in one dose, two doses or three doses or more. In particular embodiments, the one or more doses are administered after the subject is exposed to whole-body radiation. The one or more doses may be independently selected from about 1 mg/kg to about 40 mg/kg. In particular embodiments, the one or more doses are independently selected from about 10 mg/kg to about 30 mg/kg.

In certain embodiments, the subject exposed to whole-body radiation has acute radiation syndrome (ARS). The subject exposed to whole-body radiation may display symptoms of hematopoietic, gastrointestinal and/or cerebrovascular syndromes. In certain embodiments, the symptoms include one or more of anemia, infection, bleeding, nausea, vomiting, diarrhea, severe dehydration, sepsis, and petechiae.

The methods described herein may further comprise administering one or more additional treatments to the subject. In particular, the additional treatment may be selected from one or more of a blood transfusion, antibiotics and a bone marrow transplant.

### BRIEF DESCRIPTION OF THE FIGURES

FIG. 1 provides a graph illustrating the survival of CD2F1 mice after total body irradiation: control group with no post-irradiation treatment (o); 0.1 mL PBS administered subcutaneously at 4 h post-irradiation (□); 0.1 mL PBS administered subcutaneously at 24, 36, and 48 h post-irradiation (Δ); 0.1 mL 25 mg/kg ODSH administered subcutaneously at 4, 16, and 28 h post-irradiation (▪); and 0.1 mL 25 mg/kg ODSH administered subcutaneously at 24, 36, and 48 h post-irradiation (▴).

### DETAILED DESCRIPTION OF THE INVENTION

### Definitions

The phrase “radiation damage” as used herein refers to the health effects which present after exposure to high amounts of ionizing radiation. Examples of radiation damage include, but are not limited to, cell injury, tissue damage, organ dysfunction, acute radiation syndrome, and delayed radiation effects such as radiation-induced lifespan shortening, cataract development, and carcinogenesis. Radiation damage further includes any other damage relating to or caused by exposure to whole-body radiation.

A “subject”, “patient” or “host” refers to either a human or a non-human mammal.

The phrase “therapeutically effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect at a reasonable benefit/risk ratio applicable to such treatment.

“Treating” a condition or disease refers to curing as well as ameliorating at least one symptom of the condition or disease.

### Exposure to Whole-Body Radiation

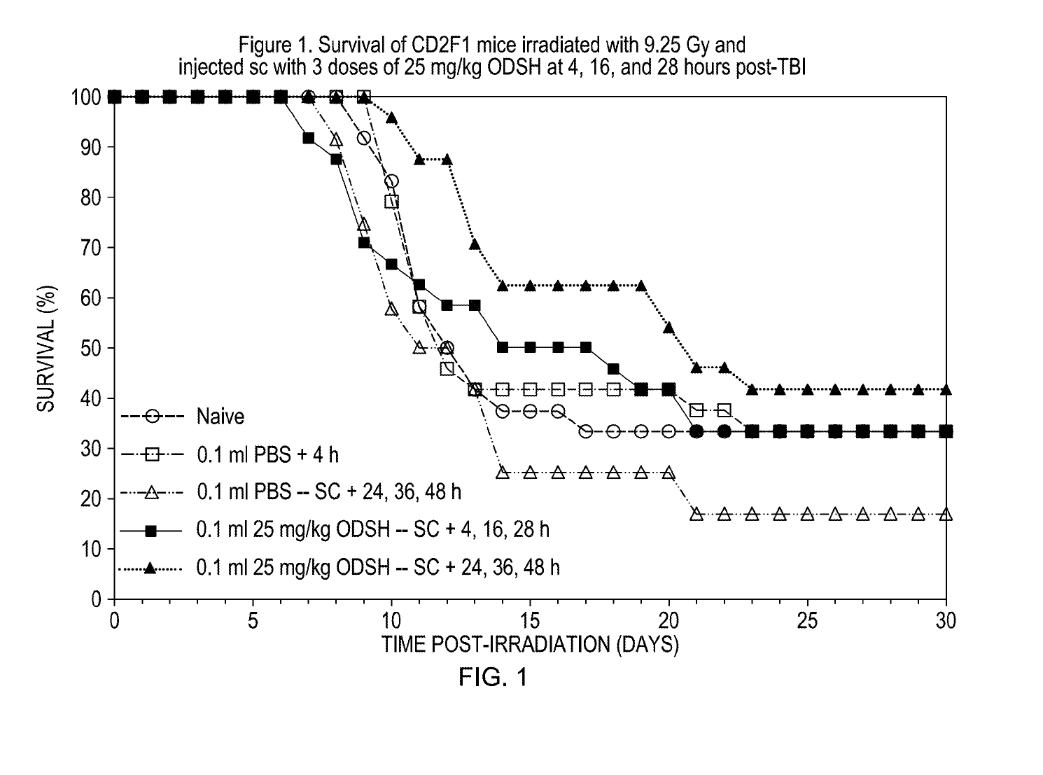

FIG. 1 presents results of a study showing the survival of CD2F1 mice that underwent whole-body irradiation with a lethal dose of radiation, 9.25 Gy. The naïve mice (o) received no treatment following irradiation while other mice received either ODSH (a heparinoid that is substantially desulfated at 2-O and 3-O positions, further described herein below) or phosphate buffered saline (PBS) in one or more doses following irradiation. Mice treated with 0.1 mL 25 mg/kg ODSH post-irradiation at 24, 36 and 48 hours, (▴), showed the greatest percentage survival over the course of the post-irradiation period in comparison to naïve mice and mice treated with PBS. In particular, at day 10 post-irradiation, 95% of the mice treated with ODSH at 24, 36 and 48 hours were still alive while half of the naïve mice had died.

In a first aspect, methods are provided for treating or preventing radiation damage in a subject exposed to whole-body radiation, comprising administering to a subject exposed to whole-body radiation a therapeutically or prophylactically effective amount of a low-anticoagulant heparinoid. In an additional aspect, methods for extending the life of a subject exposed to whole-body radiation are presented, the methods comprising administering a therapeutically or prophylactically effective amount of a low-anticoagulant heparinoid to the subject exposed to whole-body radiation.

A subject suitable for treatment by the methods described herein may be exposed to dangerous doses of radiation through contamination and/or irradiation. Contamination generally involves contact with and retention of radioactive material, usually as a dust or liquid. Contamination may be external contamination, e.g., on the skin or clothing. Alternatively the contamination may be internal, which occurs when radioactive material enters the body, e.g., through ingestion, inhalation, or through breaks in the skin. Typical radionuclides involved in contamination include hydrogen-3, cobalt-60, strontium-90, cesium-137, iodine-131, radium-226, uranium-235, uranium-238, plutonium-238, plutonium-239, polonium-210, and americium-241.

In irradiation exposure, a subject is exposed to radiation but the radiation exposure occurs without the source of radiation being in contact with the person. For irradiation exposure, when the source of the radiation is removed or turned off, e.g., X-ray instrument, exposure to the radiation ends.

Exposure to radiation can be from a number of sources including natural and man-made sources. Examples of man-made sources include radiation accidents, nuclear accidents, nuclear terrorism, nuclear war, other radiological emergencies, radiation therapy, and diagnostic radiology. Examples of natural sources include cosmic radiation and radiation from the air, water and earth. Time spent at high altitudes amplifies exposure to galactic cosmic radiation and solar particle event radiation making aviators, flight crews, and astronauts, particularly susceptible to this type of exposure. Radiation therapy may include radiation treatments of cancer or radiotherapy as part of the preparative regimen for hematopoietic stem cell or bone marrow transplantation. Diagnostic radiology may include X-ray radiographing, CT scanning, and nuclear medicine imaging.

Whole-body radiation, as used herein, refers to radiation exposure of the entire body of a subject or substantially the entire body. The entire body or substantially the entire body receives radiation when the body is exposed to a radiation source and no protective measures or limited protective measures are used to protect the body from exposure to radiation. Protective measures for ionizing radiation include, for example, barriers of lead, concrete or water which provide protection from energetic particles such as gamma rays and neutrons. In particular embodiments, whole-body radiation refers to exposure of at least the brain, stomach, intestines, pelvis and sternum or portions thereof. Whole-body radiation may be radiation administered for a therapeutic or diagnostic purpose. Alternatively, whole-body radiation may be the result of an inadvertent or undesirable exposure to whole-body radiation. For example, whole-body radiation may occur through a nuclear attack or an astronaut's exposure to cosmic radiation.

The radiation a subject is exposed to may be any type of radiation; in typical embodiments, the radiation is ionizing radiation. Ionizing radiation includes subatomic particles of matter moving at relativistic speeds and electromagnetic waves on the short wavelength end of the electromagnetic spectrum, which act like energetic particles. Common particles include alpha particles, beta particles, neutrons, and various other particles such as mesons that constitute cosmic rays.

Alpha particles are energetic helium nuclei emitted by some radionuclides with high atomic numbers, e.g., plutonium, radium, uranium. Alpha particles cannot penetrate skin beyond a shallow depth (<0.1 mm). Beta particles are high-energy electrons that are emitted from the nuclei of unstable atoms, e.g., cesium-137, iodine-131. These particles can penetrate more deeply into skin (1 to 2 cm) and cause both epithelial and subepithelial damage. Neutrons are electrically neutral particles emitted by a few radionuclides, e.g., californium-252, and produced in nuclear fission reactions, e.g., in nuclear reactors. Neutrons can penetrate deeply into tissues (>2 cm), where they collide with the nuclei of stable atoms, resulting in emission of energetic protons, alpha and beta particles, and gamma radiation.

Gamma radiation and x-rays are electromagnetic radiation, i.e., photons, of very short wavelength that can penetrate deeply into tissue (many centimeters). While some photons deposit all their energy in the body, other photons of the same energy may only deposit a fraction of their energy and others may pass completely through the body without interacting.

Because of these characteristics, alpha and beta particles cause the most damage when the radioactive atoms that emit them are within the body, i.e., internal contamination, or, in the case of beta-emitters, directly on the body; only tissue in close proximity to the radionuclide is affected. Gamma rays and x-rays can cause damage distant from their source and are typically responsible for acute radiation syndromes (ARS).

Conventional units of measuring radiation include the roentgen, rad, and rem. The roentgen (R) is a unit of exposure measuring the ionizing ability of x-ray or gamma radiation in air. The radiation absorbed dose (rad) is the amount of that radiation energy absorbed per unit of mass. Because biologic damage per rad varies with radiation type, e.g., it is higher for neutrons than for x-ray or gamma radiation, the dose in rad is corrected by a quality factor; the resulting effective dose unit is the roentgen equivalent in man (rem). In the scientific literature, SI units are used, in which the rad is replaced by the gray (Gy) and the rem by the sievert (Sv); 1 Gy=100 rad and 1 Sv=100 rem. The rad and rem (and hence Gy and Sv) are essentially equal when describing gamma or beta radiation.

When ionizing radiation is emitted by or absorbed by an atom, it can liberate an atomic particle, typically an electron, proton, or neutron, but sometimes an entire nucleus, from the atom. Such an event can alter chemical bonds and produce ions, usually in ion-pairs, that are especially chemically reactive. This greatly magnifies the chemical and biological damage per unit energy of radiation because chemical bonds will be broken in this process.

In certain embodiments, a subject is exposed to whole-body radiation at a dose rate of about 0.1 Gy/min or greater. For example, the subject may be exposed to whole-body radiation at a dose rate of about 0.2 Gy/min or greater, about 0.3 Gy/min or greater, about 0.4 Gy/min or greater, about 0.5 Gy/min or greater, about 0.6 Gy/min or greater, about 0.7 Gy/min or greater, about 0.8 Gy/min or greater, about 0.9 Gy/min or greater, or about 1.0 Gy/min or greater. In particular, the subject may be exposed whole-body radiation at a dose rate of about 0.5 Gy/min or greater.

The subject's exposure to radiation may occur over a time period of days or weeks or alternatively over a period of one day or less. For example, the radiation exposure may occur over a time period of about 10 hours or less, such as about 8 hours or less, such as about 7 hours or less, such as about 6 hours or less, such as about 5 hours or less, such as about 4 hours or less, such as about 3 hours or less, such as about 2 hours or less, such as about 1 hour or less. In particular, the subject's exposure to radiation occurs over a time period of about 2 hours or less or about 1 hour or less.

The subject may have a whole-body absorbed dose of radiation of about 2 Gy or greater. Whole-body absorbed dose, as used herein, refers to the energy deposited in a subject by ionizing radiation per unit mass. It is equal to the energy deposited per unit mass of medium, which may be measured as joules per kilogram and represented by the equivalent SI unit, gray (Gy). The absorbed dose depends not only on the incident radiation but also on the absorbing material: a soft X-ray beam may deposit four times more dose in bone than in air, or none at all in a vacuum. In particular embodiments, the whole-body absorbed dose is about 6 Gy or greater or about 8 Gy or greater. The whole-body absorbed dose may be from about 1 Gy to about 2 Gy, about 2 Gy to about 6 Gy, about 6 Gy to about 8 Gy, about 8 Gy to about 30 Gy, or greater than 30 Gy. The whole-body radiation exposure may occur through irradiation or contamination.

### Low-Anticoagulant Heparinoids

In the methods described herein, the subject exposed to whole-body radiation is administered a therapeutically or prophylactically effective amount of a low-anticoagulant heparinoid.

“Low-anticoagulant heparinoids”, as used herein, are linear glycosaminoglycan polymers made up of alternating or repeating iduronic acid and glucosamine units bearing O-sulfate, N-sulfate, and N-acetyl substitutions. Preferably, low-anticoagulant heparinoids for use in the methods described herein are polymers having an average molecular weight of at least about 8 kDa, for example having an average molecular weight ranging from about 8 kDa to about 15 kDa. In certain embodiments, the low-anticoagulant heparinoids have an average molecular weight of greater than about 8 kDa. More preferably, low-anticoagulant heparinoids for use in the methods described herein have an average molecular weight that ranges in size from about 11 kDa to about 13 kDa.

The low-anticoagulant heparinoids may have an average molecular weight from about 2 kDa to about 15 kDa. In certain embodiments, the low-anticoagulant heparinoids have an average molecular weight of at least about 2 kDa, at least about 3 kDa, at least about 4 kDa, at least about 5 kDa, at least about 6 kDa, or at least about 7 kDa. In certain embodiments, the low- anticoagulant heparinoids have an average molecular weight of less than about 15 kDa, less than about 14 kDa, less than about 13 kDa, less than about 12 kDa, less than about 11 kDa, less than about 10 kDa, or less than about 9 kDa. In some embodiments, the average molecular weight of the low-anticoagulant heparinoid is selected from about 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa or a range including any of these values as endpoints. Molecular weight of heparinoids can be determined by high performance size exclusion chromatography as is known in the art. See, e.g., Lapierre et al., 1996, Glycobiology 6(3):355-366, at page 363; Fryer et al., 1997, J. Pharmacol. Exp. Ther. 282: 208-219, at page 209.

The low-anticoagulant heparinoids used in the methods described herein have reduced anticoagulant activity or are substantially non-anticoagulant. Low anticoagulant heparinoids have no more than 40% of the anti-coagulant activity of an equal weight of unfractionated heparin. For example, the low-anticoagulant heparinoid has no more than 35%, no more than 30%, no more than 20%, even no more than 10%, 9%, 8%, 7%, 6%, 5%, 4%, 3%, 2% or 1% of the anti-coagulant activity of an equal weight of unfractionated heparin. In certain embodiments, the low-anticoagulant heparinoids interact with Platelet Factor 4 (PF4), for example, the heparinoids bind to PF4.

Anticoagulant activity can be determined using assays known in the art. In certain embodiments, anticoagulant activity is determined by activated partial thromboplastin time (aPTT) assay. In some embodiments, anticoagulant activity is determined by assay of prothrombin time. In particular embodiments, anticoagulant activity is determined by anti-Xₐ activity. In a variety of embodiments, anticoagulant activity is determined by clotting assay. In some embodiments, anticoagulant activity is determined by amidolytic assays. In certain embodiments, anticoagulant activity is determined by the USP assay. See, e.g., U.S. Pat. No. 5,668,118, Example IV; Fryer et al., 1997, J. Pharmacol. Exp. Ther. 282: 208-219, at page 209; Rao et al., 2010, Am. J. Physiol. 299:C97-C110, at page C98; United States Pharmacopeia Convention 1995 (for USP anti-coagulant assay and amidolytic assay).

A low-anticoagulant heparinoid used in the methods described herein is low-anticoagulant in at least one of the above-described assays. In certain embodiments, the low-anticoagulant heparinoid used in the methods described herein is low-anticoagulant in more than one of the above-described assays.

In a variety of embodiments, the substantially anti-coagulant heparinoid is one which exhibits substantially reduced anti-Xₐ activity, which can be determined in an assay carried out using plasma treated with Russell viper venom.

In specific embodiments, the low-anticoagulant heparinoid used in the methods described herein is ODSH, further described below. ODSH has been demonstrated to exhibit less than 9 U of anti-coagulant activity/mg in the USP anti-coagulant assay (e.g., 7±0.3 U), less than 5 U of anti-Xₐ activity/mg (e.g., 1.9±0.1 U/mg) and less than 2 U of anti-IIₐ activity/mg (e.g., 1.2±0.1 U/mg). Unfractionated heparin has an activity of 165-190 U/mg in all three assays. See Rao et al., 2010, Am. J. Physiol. 299:C97-C110, page C101. In addition, ODSH has a low affinity for anti-thrombin III (Kd ˜339 μM or 4 mg/ml vs. 1.56 μM or 22 μg/ml for unfractionated heparin), consistent with the observed low level of anti-coagulant activity, measured as described in Rao et al., supra, at page C98.

In typical embodiments, the low-anticoagulant heparinoids are partially desulfated. Preferably, the low-anticoagulant heparinoids are substantially desulfated at the 2-O position of α-L-iduronic acid (referred to herein as the “2-O position”) and/or desulfated at the 3-O position of D-glucosamine-N-sulfate (6-sulfate) (referred to herein as the “3-O position”). In some embodiments, the low-anticoagulant heparinoids are at least 85%, at least 90%, at least 95%, at least 96%, at least 97%, at least 98%, or at least 99% desulfated at the 2-O position. In selected embodiments, the low-anticoagulant heparinoids are at least 99% desulfated at the 2-O position. In some embodiments, the low-anticoagulant heparinoids are at least 85%, at least 90%, at least 95%, at least 96%, at least 97%, at least 98%, or at least 99% desulfated at the 3-O position. In selected embodiments, the low-anticoagulant heparinoids are at least 99% desulfated at the 3-O position. In some embodiments, the low-anticoagulant heparinoids are at least 85%, at least 90%, at least 95%, at least 96%, at least 97%, at least 98%, or at least 99% desulfated at both the 2-O position and the 3-O position. In selected embodiments, the low-anticoagulant heparinoids are at least 99% desulfated at the 2-O position and the 3-O position.

In typical embodiments, the low-anticoagulant heparinoid comprises substantially N-sulfated and 6-O sulfated D-glucosamine. In some embodiments, the carboxylates on α-L-iduronic acid sugars of low-anticoagulant heparinoid are substantially intact.

An exemplary low-anticoagulant heparinoid is substantially 2-O, 3-O desulfated heparin, referred to herein as ODSH. ODSH for use in the above-described methods can be prepared from bovine or porcine heparin. In an exemplary method of preparing ODSH from porcine heparin, ODSH is synthesized by cold alkaline hydrolysis of USP porcine intestinal heparin, which removes the 2-O and 3-O sulfates, leaving N- and 6-O sulfates on D-glucosamine sugars and carboxylates on α-L-iduronic acid sugars substantially intact. Fryer, A. et al., 1997, J. Pharmacol. Exp. Ther. 282: 208-219. Using this method, ODSH can be produced with an average molecular weight of about 11.7±0.3 kDa.

Methods for the preparation of 2-O, 3-O desulfated heparin may also be found, for example, in U.S. Pat. Nos. 5,668,118, 5,912,237, and 6,489,311, and WO 2009/015183, the contents of which are incorporated herein in their entirety, and in U.S. Pat. Nos. 5,296,471, 5,969,100, and 5,808,021.

### Pharmaceutical Compositions

In typical embodiments, the low-anticoagulant heparinoid is administered in the form of a pharmaceutical formulation or composition. Pharmaceutical compositions, suitable for administration to subjects, may optionally include additional active and/or therapeutic agents, as is known in the art. See Remington: The Science and Practice of Pharmacy, 21^{st} Ed. (2005), Lippincott Williams & Wilkins, incorporated herein by reference. The formulations will typically include one or more pharmaceutically acceptable carriers, excipients, or diluents. The specific carriers, excipients, and/or diluents used will depend on the desired mode of administration.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the subject. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In various embodiments, the pharmaceutical composition is in the form of a sterile, non-pyrogenic, fluid composition.

### Modes of Administration

The pharmaceutical compositions for use in the methods described herein can be formulated for administration to subjects by a variety of routes, including intranasally, by inhalation, intramuscularly, intraperitoneally, and parenterally, including intravenously or subcutaneously. The pharmaceutical compositions can be formulated in volumes and concentrations suitable for bolus administration, for continuous infusion, or for subcutaneous administration. In preferred embodiments, the low-anticoagulant heparinoid is administered parenterally, either intravenously, subcutaneously, or both intravenously and subcutaneously.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

Pharmaceutical compositions can be conveniently presented in unit dosage forms which contain a predetermined amount of low-anticoagulant heparinoid. In various embodiments, unit dosage forms of low-anticoagulant heparinoid for use in the methods described herein contain 1 mg to 1 g, or 5 mg to 500 mg of low-anticoagulant heparinoid.

Low-anticoagulant heparinoids can be administered in the methods described herein by a variety of routes, as noted above. In particular embodiments, the low-anticoagulant heparinoid is administered intravenously and/or subcutaneously.

### Schedule of Administration

In certain embodiments, low-anticoagulant heparinoid is administered to a subject following the subject's exposure to whole-body radiation. The subject may be administered one or more doses of low-anticoagulant heparinoid within about 60 hours following exposure to whole-body radiation. The phrase “following exposure to whole-body radiation” refers generally to the period of time beginning after a subject's exposure to irradiation or contamination. For example, a subject is exposed to high doses of ionizing radiation over the course of 1 hour and at the completion of the hour, the period following exposure to radiation commences. In certain embodiments, the subject's exposure may be contamination, such as internal contamination, wherein the exposure to radiation continues until the radiation is removed or decays. In the event of continuing exposure to radiation such as with internal contamination, the period following exposure to whole-body radiation is defined herein as the period following initial exposure to the contaminant. For example, a subject ingests a radionuclide and the period following exposure to whole-body radiation commences at the time of ingestion.

The low-anticoagulant heparinoid may be administered at about 2 hours or more after exposure to whole-body radiation. The low-anticoagulant heparinoid may be administered in one or more doses about 2 hours or more following exposure to whole-body radiation. In particular, the low-anticoagulant heparinoid may be administered in three or more doses about 2 hours or more following exposure to whole-body radiation. For example, the low-anticoagulant heparinoid may be administered at about 4 hours, about 16 hours, and about 28 hours after whole-body radiation exposure.

The low-anticoagulant heparinoid may be administered at about 20 hours or more after whole-body radiation exposure. In particular, the low-anticoagulant heparinoid may be administered in two or more doses, the first dose administered at about 20 hours or more after whole-body radiation exposure. For example, the low-anticoagulant heparinoid may be administered at about 24 hours, about 36 hours and about 48 hours after whole-body radiation exposure.

In certain embodiments, the subject receives one dose of low-anticoagulant heparinoid for approximately every 12 hour interval following radiation exposure, e.g., one dose in the first 12 hours following radiation exposure, a second dose in hours 13-24 following radiation exposure, etc. Dosing of low-anticoagulant heparinoid may continue for multiple days or weeks following radiation exposure such as 2 days or more, 3 days or more, 4 days or more, 5 days or more, 6 days or more, 1 week or more, 2 weeks or more, 3 weeks or more or 4 weeks or more.

The low-anticoagulant heparinoid may be administered continuously over a period of time such as from about 2 to 10 hours. For example, the subject may receive an intravenous infusion of low-anticoagulant heparinoid continuously over a period of about 2 hours or more, about 3 hours or more, about 4 hours or more, about 5 hours or more, about 6 hours or more, about 7 hours or more, or about 8 hours or more. The continuous administration of low-anticoagulant heparinoid may occur before, after and/or during exposure to radiation. There may be multiple session of continuous administration such as 3 or more sessions of continuous administration. For example, a subject may receive a session of continuous administration for 3 hours or more for each 24 hour period following exposure to whole-body radiation up to 1-2 weeks following exposure.

The low-anticoagulant heparinoid may be administered prior to exposure to whole-body radiation. In certain embodiments, the low-anticoagulant heparinoid is administered to subjects who must enter a known radiation zone, such as a damaged nuclear reactor or its environs, patients who are scheduled for radiation therapy, patients who are scheduled for diagnostic therapy, subjects scheduled for space travel, and where possible, those who may be exposed due to expected nuclear attack. In such embodiments, treatments may include a single dose or multiple doses of low-anticoagulant heparinoid prior to whole-body radiation exposure.

The low-anticoagulant heparinoid may be administered during exposure to whole-body radiation exposure. Administration during whole-body radiation exposure may be helpful for subjects undergoing radiotherapy for stem cell or bone marrow transplants. A subject may receive infusions of low-anticoagulant heparinoid during radiotherapy treatments to prevent or ameliorate radiation damage to tissues that are not the intended target of the radiotherapy.

A subject may be administered a low-anticoagulant heparinoid any one or more of before, during, and after exposure to whole-body radiation. A subject may be administered a low-anticoagulant heparinoid both before and after exposure to whole-body radiation exposure. For example, a marine entering a war zone receives a preventative amount of low-anticoagulant heparinoid prior to and following exposure to whole-body radiation exposure. A pregnant woman may be administered a low-anticoagulant heparinoid prior to and following radiotherapy, so as to mitigate radiation damage in the fetus and in maternal tissues not the intended target of the radiotherapy.

Low-anticoagulant heparinoids may be administered to the subject in an amount sufficient or effective to provide a therapeutic benefit, i.e., a therapeutically effective amount, and/or a preventative benefit, i.e., prophylactically effective amount. The therapeutically effective amount and prophylactically effective amount depend in part on the amount of whole-body radiation the subject is exposed to or will be exposed to, the extent of radiation damage and other characteristics of the subject to be treated, e.g., age, size, etc.

The one or more doses of low-anticoagulant heparinoid may be independently selected from different low-anticoagulant heparinoids. For example, the subject may receive one or more doses of ODSH as well as one or more doses of a different low-anticoagulant heparinoid.

One or more doses of low-anticoagulant heparinoid may be independently selected from about 1 mg/kg to about 40 mg/kg. In particular, one or more doses may be independently selected from about 10 mg/kg to about 30 mg/kg.

### Treating and Preventing Radiation Damage

The methods described herein may be used to treat or prevent radiation damage for a subject with acute radiation syndrome (ARS). ARS is a constellation of health effects which present within 24 hours of whole-body exposure to high amounts of ionizing radiation. ARS is generally divided into three main presentations: hematopoietic syndrome, gastrointestinal syndrome and cerebrovascular syndrome. Subjects exposed to high levels of whole-body radiation will generally experience these syndromes in varying degrees dependent upon their dosage of radiation.

The hematopoietic syndrome is the dominant manifestation after whole-body doses of about 1 to 6 Gy and consists of a generalized pancytopenia. Bone marrow stem cells are significantly depleted. As the cells in circulation die by senescence, they are not replaced in sufficient numbers, resulting in pancytopenia. Risk of various infections is increased as a result of the neutropenia and decreased antibody production. Petechiae and mucosal bleeding result from thrombocytopenia. Anemia develops slowly, because preexisting red blood cells have a longer life span than white blood cells and platelets. Survivors have an increased incidence of radiation-induced cancer, including leukemia.

The gastrointestinal syndrome is the dominant manifestation after whole-body doses of about 6 to 30 Gy. GI mucosal cell death, caused by the radiation, is followed by intractable nausea, vomiting, and diarrhea, which lead to severe dehydration and electrolyte imbalances, diminished plasma volume, and vascular collapse. Necrosis of the intestine may also occur, predisposing to bacteremia and sepsis. Subjects receiving >10 Gy may have cerebrovascular symptoms suggesting a lethal dose. Survivors also have the hematopoietic syndrome. In certain embodiments, the methods of treating or preventing radiation damage described herein may be particularly suited for subjects with radiation damage to the gastrointestinal system.

The cerebrovascular syndrome, the dominant manifestation of extremely high whole-body doses of radiation (>30 Gy), is generally fatal. It presents with neurological symptoms such as dizziness, headache, or decreased level of consciousness, occurring within minutes to a few hours, and with an absence of vomiting. Subjects develop tremors, seizures, ataxia, and cerebral edema and often die within hours to 1 or 2 days.

In a variety embodiments, administration of a therapeutically or prophylactically effective dose of low-anticoagulant heparinoid treats or prevents symptoms other than myelosuppression. In certain embodiments, administration of a therapeutically or prophylactically effective dose of low-anticoagulant heparinoid treats or prevents symptoms other than thrombocytopenia. In certain embodiments, administration of a therapeutically or prophylactically effective dose of substantially low-anticoagulant heparinoid treats or prevents symptoms other than neutropenia.

Methods described herein may be used to extend the life of subjects exposed to whole-body radiation. For example, the administration of a low-anticoagulant heparinoid may extend the life of a subject exposed to a lethal dose of whole-body radiation by about 1 day or more, about 2 days or more, about 3 days or more, about 4 days or more, or about a week or more. In certain embodiments, the methods described herein may be used to extend the life of the subject until other forms of treatment may be administered.

In certain embodiments, the methods further comprise one or more additional treatments. The one or more additional treatments may be selected from one or more of a blood transfusion, antibiotics and a bone marrow transplant. For example, a subject may receive one or more doses of a low-anticoagulant heparinoid, a blood transfusion and antibiotics.

### EXAMPLES

### Example 1

### Survival of CD2F1 Mice Irradiated with 9.25 Gy and Injected Subcutaneously with 25 mg/kg ODSH Post-TBI (Total Body Irradiation)

This experiment demonstrates that administration of ODSH at intervals following total body irradiation improves survival of mice relative to PBS or the control group without therapy. (see FIG.1)

### Materials and Methods:

CD2F1 male mice (Batch #7586 DOB Dec. 23, 2012) were weighed and animals outside ±20% of the mean weight were excluded. Mice that were within ±20% of the mean weight were randomized into groups of eight animals per box. There were 24 animals per treatment group. The animals received radiation at a dose rate of 0.6 Gy/min in the AFRRI Cobalt 60 gamma radiation facility. Animals were irradiated in Lucite boxes (8 animals/box) and arranged in an array (dosimetry Feb. 25, 2010) using plastic racks. Animals were restrained for no more than 60 min and returned to cages at the end of the irradiation period.

Post-TBI, animals were untreated (naïve) or treated subcutaneously with either phosphate buffered solution (PBS) or ODSH (2-O, 3-O desulfated heparin). Animals treated with PBS were subcutaneously administered 0.1 mL of PBS at either 4 h post-TBI or 24, 36 and 48 h post-TBI. Animals treated with ODSH were subcutaneously administered 0.1 mL of 25 mg/kg at 4, 16 and 28 h post-TBI or 24, 36, and 48 h post-TBI. The animals were monitored daily (twice a day when necessary) for 30 days and euthanized at the completion of the observational period.

Results are shown in FIG. 1, and demonstrate that mice treated with 0.1 mL 25 mg/kg ODSH post-irradiation at 24, 36 and 48 hours, (▴), showed the greatest percentage survival over the course of the post-irradiation period in comparison to naïve mice and mice treated with PBS. In particular, at day 10 post-irradiation, 95% of the mice treated with ODSH at 24, 36 and 48 hours were still alive while half of the naïve mice had died.

### EQUIVALENTS

The present disclosure provides, inter alia, methods of treating and preventing radiation damage and extending life for subjects exposed to whole-body radiation. While specific embodiments of these methods have been discussed, the above specification is illustrative and not restrictive. Many variations will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

1. A method of treating radiation damage, preventing radiation damage or extending the life of subject exposed to whole-body radiation, comprising administering to a subject exposed to whole-body radiation a therapeutically or prophylactically effective amount of a low-anticoagulant heparinoid.

2. (canceled)

3. The method of claim 1, wherein the low-anticoagulant heparinoid has an average molecular weight of about 8 kDa to about 15 kDa.

4. The method of claim 3, wherein the low-anticoagulant heparinoid is desulfated or substantially desulfated at the 2-O position or the 3-O position.

5. The method of claim 1 or 3, wherein the low-anticoagulant heparinoid is desulfated or substantially desulfated at the 2-O and 3-O positions.

6. The method of claim 1, wherein the low-anticoagulant heparinoid is administered parenterally.

7. The method of claim 6, wherein the low-anticoagulant heparinoid is administered intravenously or subcutaneously.

8. The method of claim 1, wherein the subject is administered the low-anticoagulant heparinoid following exposure to whole-body radiation.

9.-10. (canceled)

11. The method of claim 1, wherein the subject is administered the low-anticoagulant heparinoid prior to exposure to whole-body radiation.

12. The method of claim 1, wherein the subject is exposed to whole-body radiation at a dose rate of about 0.1 Gy/min or greater.

13.-18. (canceled)

19. The method of claim 1, wherein the low-anticoagulant heparinoid is administered in one or more doses.

20. The method of claim 19, wherein the one or more doses are independently selected from about 1 mg/kg to about 40 mg/kg.

21. (canceled)

22. The method of claim 1, wherein the subject exposed to whole-body radiation has acute radiation syndrome.

23. The method of claim 1, wherein the subject exposed to whole-body radiation displays symptoms of hematopoietic, gastrointestinal and/or cerebrovascular syndromes.

24. The method of claim 23, wherein symptoms include one or more of anemia, infection, bleeding, nausea, vomiting, diarrhea, severe dehydration, sepsis, and petechiae.

25. The method of claim 1, further comprising administering one or more additional treatments selected from a blood transfusion, antibiotics and a bone marrow transplant.

26. (canceled)

27. The method of claim 4, wherein the low-anticoagulant heparinoid is substantially desulfated at the 2-O position.

28. The method of claim 27, wherein the substantially 2-O desulfated heparinoid is at least 85% desulfated at the 2-O position.

29. The method of claim 28, wherein the substantially 2-O desulfated heparinoid is at least 90% desulfated at the 2-O position.

30. The method of claim 29, wherein the substantially 2-O desulfated heparinoid is at least 95% desulfated at the 2-O position.

31. The method of claim 4, wherein the low-anticoagulant heparinoid is substantially desulfated at the 3-O position.

32. The method of claim 31, wherein the substantially 3-O desulfated heparinoid is at least 85% desulfated at the 3-O position.

33. The method of claim 32, wherein the substantially 3-O desulfated heparinoid is at least 90% desulfated at the 3-O position.

34. The method of claim 33, wherein the substantially 3-O desulfated heparinoid is at least 95% desulfated at the 3-O position.

35. The method of claim 5, wherein the substantially 2-O and 3-O desulfated heparinoid is at least 85% desulfated at the 2-O and 3-O positions.

36. The method of claim 35, wherein the substantially 2-O and 3-O desulfated heparinoid is at least 90% desulfated at the 2-O and 3-O positions.

37. The method of claim 36, wherein the substantially 2-O and 3-O desulfated heparinoid is at least 95% desulfated at the 2-O and 3-O positions.
